(19) United States
(12) Patent Application Publication
(10) Pub. No.: US 2017/0071525 A1
(43) Pub. Date: Mar. 16, 2017

(54) DEVICE OF DROWSINESS DETECTION AND ALARM AND METHOD OF THE SAME

(71) Applicant: NATIONAL CHIAO TUNG UNIVERSITY, HSINCHU (TW)

(72) Inventors: Chin-Teng LIN, HSINCHU (TW); Yi-Hsin YU, HSINCHU (TW); Li-Wei KO, HSINCHU (TW)

(21) Appl. No.: 15/267,564

(22) Filed: Sep. 16, 2016

(30) Foreign Application Priority Data
Sep. 16, 2015 (TW) 104130655

(51) Int. Cl. A61B 5/18; A61B 5/00; G08B 21/06; A61B 5/0476

(52) U.S. Cl. CPC A61B 5/18; A61B 5/0476; A61B 5/7278; A61B 5/6893; G08B 21/06

(57) ABSTRACT

A device of drowsiness detection and alarm includes a headrest, at least one brainwave sensor, a computing unit, a controlling unit and an accessory unit. The brainwave sensor is disposed on the headrest and configured to contact with a user's head and detect a brainwave signal. The computing unit receives the brainwave signal from the brainwave sensor and calculates according to the brainwave signal to obtain a fatigue index. The controlling unit controls the accessory unit to activate according to the fatigue index. Therefore, when the user is drowsy and gradually loses consciousness, the accessory unit can immediately alarm the user and allow the user stay awake to avoid the occurrence of accidents. A method of drowsiness detection and alarm is also disclosed.

### CROSS REFERENCE TO RELATED APPLICATIONS

This Non-provisional application claims priority under 35 U.S.C. §119(a) on Patent Application No(s). 104130655 filed in Taiwan, Republic of China on Sep. 16, 2015, the entire contents of which are hereby incorporated by reference.

### BACKGROUND OF THE INVENTION

Field of Invention

The present invention relates to a device and method of drowsiness detection and alarm, which can alarm the user and allow the user stay awake when the user is drowsy and gradually loses consciousness.

Related Art

As the developments of technologies, various kinds of vehicles such as cars, buses, motorcycles and bicycles are invented to provide the conveniences in transportation and traveling. However, the wounded and dead numbers of the drivers and passengers of the vehicles are increasing due to the occurrence of accidents. One of the major reasons to cause the accidents is that the vehicle drivers are drowsy and lose consciousness.

In order to avoid the occurrence of accidents caused by the physical problems of the drivers, a proper physical detector is, in the recent years, installed in the vehicle for detecting the body temperature, blood pressure, pulse and blood sugar of the driver. For example, the sensors for detecting heart beats or blood pressure are distributed on the body of the driver for monitoring the physical statuses of the driver. When the detection indicates that the physical statuses of the driver are abnormal, it can alert the driver or send a message to the remote monitoring center for the emergency response.

However, the above technology still has the following drawbacks in practice.

It is complicated and time consuming to attach a plurality of sensors on the body of the driver for detecting the body temperature, blood pressure, pulse or blood sugar. Besides, the cost for the above equipment is very high, so it is hard to commercialization.

It is desired to solve the above problem and drawbacks.

### SUMMARY OF THE INVENTION

In view of the foregoing, an objective of the invention is to provide a device and method of drowsiness detection and alarm, which can alarm the user and allow the user stay awake when the user is drowsy and gradually loses consciousness.

The present invention is to utilize the brainwave sensors of the headrest to detect the brainwave of the user for recognizing the status of the user, followed by determining whether to activate the accessory unit to alert the user or to allow the user stay awake, thereby avoiding the occurrence of accidents.

To achieve the above objective, the present invention discloses a device of drowsiness detection and alarm, which includes a headrest, at least a brainwave sensor, a computing unit, a controlling unit and at least an accessory unit. The brainwave sensor is disposed on the headrest, and the brainwave sensor, computing unit, controlling unit and accessory unit are connected with each other. When the user's head lays on the headrest so as to contact with the brainwave sensors, the brainwave sensors can detect the brainwave signal and then transmit the detected brainwave signal to the computing unit. The computing unit calculates according to the received brainwave signal to obtain a fatigue index. When the fatigue index reaches a preset value, the controlling unit controls the accessory unit to activate so as to provide an alert such as a sound, a flash, an electric shock, a vibration, water or the likes. Thus, the user can be alerted or awaked before falling sleep or losing consciousness, thereby avoiding the occurrence of accidents.

In one embodiment, the computing unit includes a database for storing human brainwave information, and a judgement unit for comparing the brainwave signal detected by the brainwave sensor with the human brainwave information stored in the database to obtain the fatigue index.

In one embodiment, the accessory unit is a speaker, a lighting device, an electric shock device, a vibration device, a watering device, a massage device, or a portable device.

In one embodiment, the headrest is a seat headrest of a vehicle.

In addition, the present invention also discloses a method of drowsiness detection and alarm, which includes the following steps of: (a) providing a headrest and detecting a brainwave signal of a user, wherein the user's head lays on the headrest and contacts with a brainwave sensor disposed on the headrest; (b) receiving the brainwave signal from the brainwave sensor by a computing unit and calculating according to the brainwave signal to obtain a fatigue index; (c) when the fatigue index reaches a preset value, outputting a message to a controlling unit; and (d) controlling an accessory unit to activate according to the fatigue index by the controlling unit.

In one embodiment, the step (b) obtains the fatigue index by comparing the brainwave signal with human brainwave information.

In one embodiment, a judgement unit determines whether the fatigue index reaches the preset value, and the accessory unit is activated according to the comparing result.

In one embodiment, the accessory unit is a speaker, a lighting device, an electric shock device, a vibration device, a watering device, a massage device, or a portable device.

In one embodiment, the headrest is a seat headrest of a vehicle.

As mentioned above, the device and method of drowsiness detection and alarm of the invention can overcome the drawbacks of the conventional art, such as the complicated installation steps and high cost.

### BRIEF DESCRIPTION OF THE DRAWINGS

The invention will become more fully understood from the detailed description and accompanying drawings, which are given for illustration only, and thus are not limitative of the present invention, and wherein:

FIG. 1 is a schematic diagram of a device of drowsiness detection and alarm according to an embodiment of the invention;

FIG. 2 is a block diagram of the device of drowsiness detection and alarm according to the embodiment of the invention;

FIGS. 3 and 4 are schematic diagrams showing the device of drowsiness detection and alarm in use; and

FIG. 5 is a flow chart of a method of drowsiness detection and alarm according to an embodiment of the invention.

### DETAILED DESCRIPTION OF THE INVENTION

The present invention will be apparent from the following detailed description, which proceeds with reference to the accompanying drawings, wherein the same references relate to the same elements.

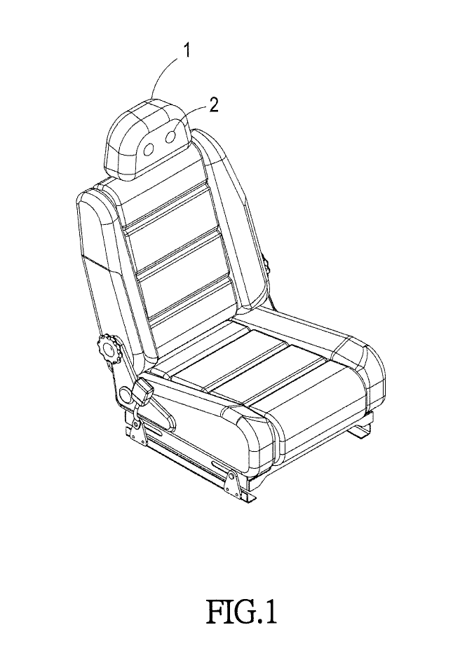

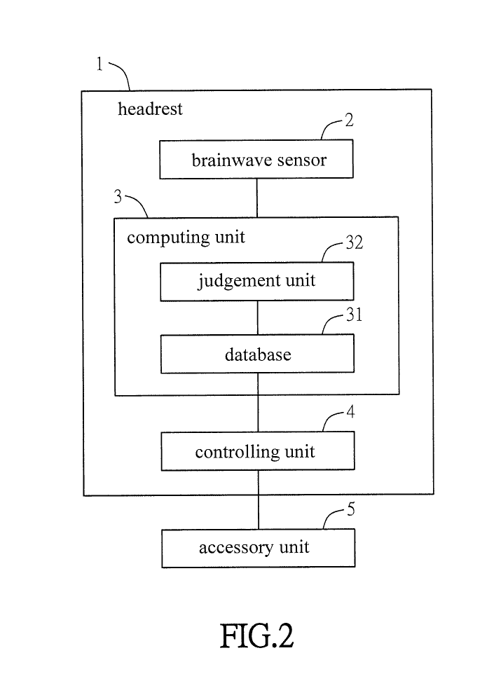

FIG. 1 is a schematic diagram of a device of drowsiness detection and alarm according to an embodiment of the invention, and FIG. 2 is a block diagram of the device of drowsiness detection and alarm according to the embodiment of the invention. Referring to FIGS. 1 and 2, the device of drowsiness detection and alarm includes a headrest 1, at least one brainwave sensor 2, a computing unit 3, a controlling unit 4, and at least one accessory unit 5.

The headrest 1 can be a seat headrest of a vehicle.

The brainwave sensor 2 is disposed on the headrest 1 and is configured to contact with a user's head for detecting a brainwave signal.

The computing unit 3 receives the brainwave signal from the brainwave sensor 2 and calculates according to the brainwave signal to obtain a fatigue index. In this embodiment, the computing unit 3 includes a database 31 and a judgement unit 32. The database 31 if configured for storing human brainwave information. The judgement unit 32 compares the brainwave signal detected by the brainwave sensor 2 with the human brainwave information stored in the database 31 to obtain the fatigue index. Herein, the judgement unit 32 produces an electroencephalography (EEG) for obtaining the fatigue index.

The controlling unit 4 connects with the computing unit 3.

The accessory unit 5 connects with the controlling unit 4, and the controlling unit 4 controls the accessory unit 5 to activate according to the fatigue index. In this embodiment, the accessory unit can be a speaker, a lighting device, an electric shock device, a vibration device, a watering device, a massage device, or a portable device.

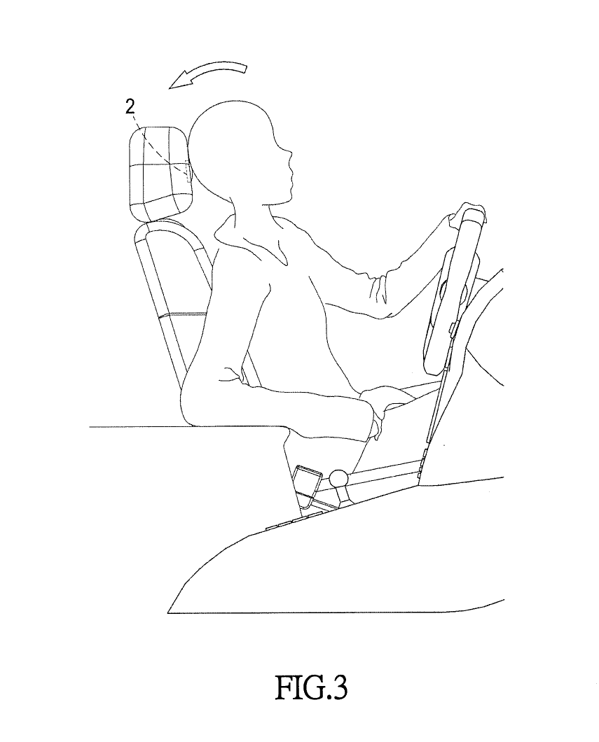

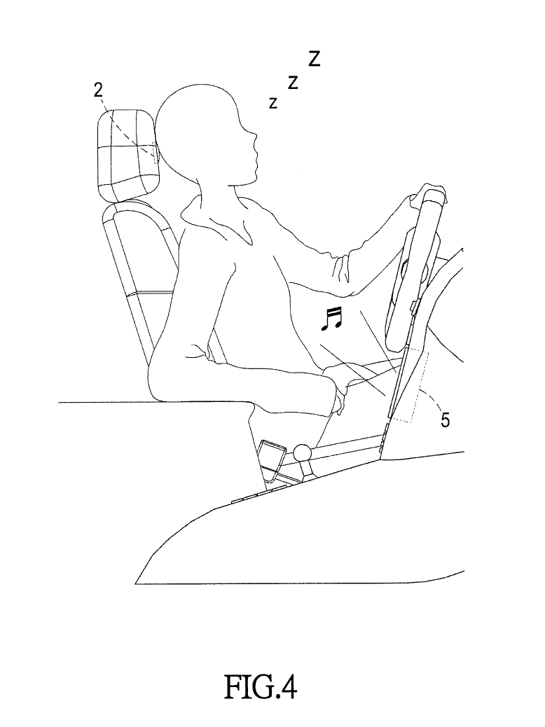

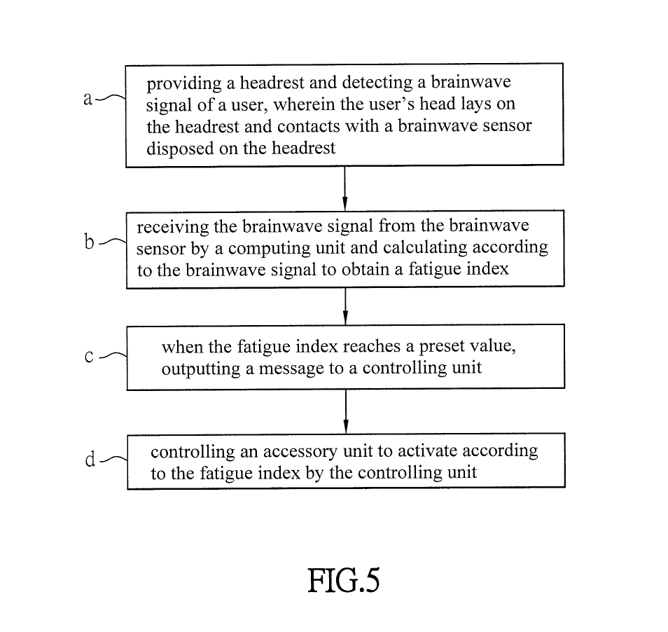

FIGS. 3 and 4 are schematic diagrams showing the device of drowsiness detection and alarm in use, and FIG. 5 is a flow chart of a method of drowsiness detection and alarm according to an embodiment of the invention. Referring to FIGS. 1 to 5, the method of drowsiness detection and alarm includes the following steps of: (a) providing a headrest and detecting a brainwave signal of a user, wherein the user's head lays on the headrest and contacts with a brainwave sensor disposed on the headrest; (b) receiving the brainwave signal from the brainwave sensor by a computing unit and calculating according to the brainwave signal to obtain a fatigue index; (c) when the fatigue index reaches a preset value, outputting a message to a controlling unit; and (d) controlling an accessory unit to activate according to the fatigue index by the controlling unit.

The method of drowsiness detection and alarm will be illustrated in details in the following example.

In the step (a), when the user feels tired, he or she may naturally lays on the headrest 1 on the seat of the vehicle. Once the user's head (occipital bone) contacts with the brainwave sensor 2 disposed on the headrest 1, the brainwave sensor 2 can detect a brainwave signal of the user.

In the step (b), after detecting the brainwave signal, the brainwave sensor 2 sends the brainwave signal to the computing unit 3, and then the computing unit 3 calculates according to the received brainwave signal to obtain a fatigue index. For example, the fatigue index is obtained by comparing the brainwave signal with human brainwave information stored in the database 31.

In the step (c), the judgement unit 32 determines whether the fatigue index reaches the preset value. Whenever the judgement unit 32 determines that the fatigue index reaches or excesses the preset value, the controlling unit 4 is activated to output a controlling signal.

In the step (d), the controlling unit 4 controls the accessory unit 5 to activate according to the fatigue index so as to alert the user. In practice, the accessory unit 5 is a speaker, a lighting device, an electric shock device, a vibration device, a watering device, a massage device, or a portable device.

In this embodiment, the headrest 1 is a seat headrest of a vehicle.

In summary, the device and method of drowsiness detection and alarm of the invention can improve the conventional art. The critical technology of the invention is stated as follow. The brainwave sensor 2 configured on the headrest 1 can detect the brainwave of the user, which is used to determine the physical status of the user. Then, the invention decides whether to activate the accessory unit 5 to alert the user or to allow the user stay awake according to the physical status of the user, thereby avoiding the occurrence of accidents.

Although the invention has been described with reference to specific embodiments, this description is not meant to be construed in a limiting sense. Various modifications of the disclosed embodiments, as well as alternative embodiments, will be apparent to persons skilled in the art. It is, therefore, contemplated that the appended claims will cover all modifications that fall within the true scope of the invention.

What is claimed is:

1. A device of drowsiness detection and alarm, comprising:
a headrest;
at least a brainwave sensor disposed on the headrest and configured to contact with a user's head for detecting a brainwave signal;
a computing unit receiving the brainwave signal from the brainwave sensor and calculating according to the brainwave signal to obtain a fatigue index;
a controlling unit connecting with the computing unit; and
at least an accessory unit connecting with the controlling unit, wherein the controlling unit controls the accessory unit to activate according to the fatigue index.

2. The device of claim 1, wherein the computing unit comprises:
a database for storing human brainwave information; and
a judgement unit comparing the brainwave signal detected by the brainwave sensor with the human brainwave information stored in the database to obtain the fatigue index.

3. The device of claim 1, wherein the accessory unit is a speaker, a lighting device, an electric shock device, a vibration device, a watering device, a massage device, or a portable device.

4. The device of claim 1, wherein the headrest is a seat headrest of a vehicle.

5. A method of drowsiness detection and alarm, comprising steps of:
(a) providing a headrest and detecting a brainwave signal of a user, wherein the user's head lays on the headrest and contacts with a brainwave sensor disposed on the headrest;
(b) receiving the brainwave signal from the brainwave sensor by a computing unit and calculating according to the brainwave signal to obtain a fatigue index;
(c) when the fatigue index reaches a preset value, outputting a message to a controlling unit; and
(d) controlling an accessory unit to activate according to the fatigue index by the controlling unit.

6. The method of claim 5, wherein the step (b) obtains the fatigue index by comparing the brainwave signal with human brainwave information.

7. The method of claim 6, wherein a judgement unit determines whether the fatigue index reaches the preset value, and the accessory unit is activated according to the comparing result.

8. The method of claim 7, wherein the accessory unit is a speaker, a lighting device, an electric shock device, a vibration device, a watering device, a massage device, or a portable device.

9. The method of claim 5, wherein the headrest is a seat headrest of a vehicle.
